(19) United States
(12) Patent Application Publication
(10) Pub. No.: US 2017/0156630 A1
(43) Pub. Date: Jun. 8, 2017

(54) SYSTEM AND METHOD FOR ADAPTIVE AND PATIENT-SPECIFIC MAGNETIC RESONANCE IMAGING

(71) Applicants: BOARD OF REGENTS OF THE UNIVERSITY OF TEXAS SYSTEM, Austin, TX (US); Koninklijke Philips N.V., Eindhoven (NL)

(72) Inventors: Refaat E. GABR, Houston, TX (US); Ponnada A. NARAYANA, Bellaire, TX (US); Amol S. PEDNEKAR, Houston, TX (US)

(21) Appl. No.: 15/373,256

(22) Filed: Dec. 8, 2016

Related U.S. Application Data
(60) Provisional application No. 62/264,601, filed on Dec. 8, 2015.

(51) Int. Cl. A61B 5/055; G01R 33/54; A61B 5/00

(52) U.S. Cl. CPC A61B 5/055; A61B 5/0013; G01R 33/543

(57) ABSTRACT

A system includes a protocol optimization system which comprises a data store containing a plurality of optimized protocol definitions, a storage device containing machine instructions, and a computing resource to execute the machine instructions. The protocol optimization system is configured to, based on an input of an optimized protocol definition, access the data store to identify a scout magnetic resonance image (MRI) scan for an MRI scanner, a processing block mapped to the identified scout MRI scan, and a diagnostic scan and provide the identified scout MRI scan to the MRI scanner. Using the processing block, the protocol optimization system analyzes a resulting image upon performance of the scout MRI scan to compute a patient tissue attribute and, using the computed patient tissue attribute, to compute an optimized parameter value for the identified diagnostic scan. The protocol optimization system provides the optimized parameter value to the MRI scanner.

### CROSS-REFERENCE TO RELATED APPLICATIONS

The present application claims priority to U.S. Provisional Patent Application No. 62/264,601, filed Dec. 8, 2015, titled “System And Method For Adaptive And Patient-Specific Magnetic Resonance Imaging,” which is hereby incorporated herein by reference in its entirety.

### STATEMENT REGARDING FEDERALLY SPONSORED RESEARCH OR DEVELOPMENT

This invention was made with government support under RO1 NS078244 awarded by The National Institute of Health. The government has certain rights in the invention.

### BACKGROUND

Imaging is widely used in modern medicine and personalized healthcare. For example, magnetic resonance is a powerful tool for the diagnosis and objective monitoring of the treatment of many diseases, including neurological, neuropsychiatric, and neurodevelopmental disorders. The MR imaging protocol may comprise a set of pulse sequences whose scan parameters are set to detect certain aspects of the pathology and function/dysfunction of the imaged tissue.

Detection of tissue pathology and function/dysfunction with MR depends on identifying contrast differences between normal and abnormal tissues as they appear in pixel values in MR imaging (MRI), or in spectral peaks in MR spectroscopy (MRS). To optimize the sensitivity and/or specificity of MRI/MRS, the contrast between the signals from different tissues should exceed the detection threshold. However, the tissue contrast depends not only on the physical/physiological properties of the tissues under investigation, but also on the scan parameters used by the experimenter. Subtle contrast differences may not be detected with sub-optimal scan protocol.

### SUMMARY

In some embodiments, a system includes an MRI scanner and a protocol optimization system which comprises a data store containing a plurality of optimized protocol definitions, a storage device containing machine instructions, and a computing resource to execute the machine instructions. The protocol optimization system is configured to, based on an input of an optimized protocol definition, access the data store to identify an attribute scout magnetic resonance image (MRI) scan for an MRI scanner, a processing block mapped to the identified attribute scout MRI scan, and a diagnostic scan and provide the identified attribute scout MRI scan to the MRI scanner. Using the processing block, the protocol optimization system analyzes a resulting image upon performance of the attribute scout MRI scan to compute a patient tissue attribute and, using the computed patient tissue attribute, to compute an optimized parameter value for the identified diagnostic scan. The protocol optimization system provides the optimized parameter value to the MRI scanner. Other embodiments include the protocol optimization system without the MRI scanner, but configured to be coupled to and to configure the MRI scanner.

### BRIEF DESCRIPTION OF THE DRAWINGS

The drawings accompanying and forming part of this specification are included to depict certain aspects of the invention. A clearer impression of the invention, and of the components and operation of systems provided with the invention, will become more readily apparent by referring to the exemplary, and therefore nonlimiting, embodiments illustrated in the drawings, wherein identical reference numerals designate the same components. Note that the features illustrated in the drawings are not necessarily drawn to scale.

FIG. 1 is a block diagram of one embodiment of an adaptive MRI system.

FIG. 2 is a flow diagram of one embodiment of a method for use with an adaptive MRI system.

FIG. 3 is a block diagram of one embodiment of an adaptive MRI system as utilized with a double inversion recover (DIR) protocol for brain imaging.

FIG. 4 is a block diagram of a computing device which may be usable to implement the techniques.

### DETAILED DESCRIPTION

As noted above, attributes of the tissue being imaged as well as various scan parameters can affect the usability of the resulting image to make a diagnosis. Traditional MRI and spectroscopy-based systems are largely based on generic protocols that are used to scan all patients (e.g., the scan parameters may be set the same for all patients). Such protocols may be based on population averages and/or assumed tissue parameters, and typically are not adapted to differences between individual patents. Although this approach provides a degree of uniformity among the data, it results in suboptimal data quality on the patient level. The current one-shoe-fits-all approach is prone to inferior image quality with the associated risk of missing important abnormalities or changes that may be too subtle to detect with a generic protocol.

The disclosed embodiments customizes or otherwise adapts the imaging protocol or pulse sequence used in an MRI to individual patients to substantially optimize the image quality for the scan obtained with respect to that patient among other goals. Some embodiments are directed to an MRI system that adapts scanning protocols to patient specific attributes determined substantially in real-time while the patient is being scanned. One or more “attribute scout” scans (also sometimes referred to as “survey” scans) of a patient may be performed to acquire data (e.g., image or other data) associated with the patient. Such data may include measurements of attributes of the tissue to be imaged (e.g., T1 and T2 relaxation times, relative proton densities, etc.) and are generally acquired and calculated rapidly (i.e., in real-time while the patient is still in the scanner). Parameter(s) pertaining to the scanner itself also can be computed from the attribute scout scan images. One or more attribute scout scan images can be analyzed to determine one or more attributes particular to the patient to use in a subsequent diagnostic scan. The subsequent scan can then be performed using the patient-specific tissue attributes and scanner parameters computed based on the attribute scout scans. In this manner, a patient-specific scanning sequence for the patient can be implemented.

The disclosed embodiments thus may allow the customization of the MR protocol or scan to each individual patient through the use of real-time data processing and parameter optimization, as well as a feedback loop for automatic adaptation of the scan parameters to the optimized parameters. For the disclosed adaptive MRI system, data analysis may be performed in real-time such that the results from the processing of one MRI acquisition (e.g., a single sequence) or attribute scout scan can be used during the same imaging session to adapt one or more subsequent acquisitions or scans. The computed patient-specific tissue attributes may be used in an automatic feedback loop to the MRI scanner to configure the scanner for one or more diagnostic scans and to trigger the performance of the diagnostic scan(s).

Accordingly, embodiments of such adaptive MRI systems may be utilized to perform patient-specific scans in a single sitting (e.g., while the patient remains in the MRI machine) thereby avoiding the necessity of summoning the patient to return for additional rounds of (often uncomfortable) imaging. Moreover, the image resulting from a scan using such optimized parameters may exhibit significant improvement over a corresponding scan performed with standard parameters. Further, the scan time may be reduced or another user-defined performance metric may be optimized.

Embodiments described herein also may provide the ability to detect problems with the image resulting from the scan. Specifically, the disclosed MRI system may analyze the image resulting from an MRI scan (scout or diagnostic) to detect sub-optimal or otherwise undesirable image quality caused by, for example, insufficient signal-to-noise ratio (SNR) or other artifacts that may be caused by patient motion or the like. An image-to-ghost ratio, a geometric distortion value, etc. additionally or alternatively may be computed. These problems may be detected while the patient is still within the MRI scanner. Because the problem is detected with the patient still available and present in the machine, the scan can easily be repeated with minimal inconvenience to the patient. Summoning the patient for additional rounds of imaging may be avoided.

The disclosed MRI system also may automatically identify and communicate the need for additional scans or suggest appropriate protocols or scans for improved diagnosis. The disclosed system can evaluate the need for additional scans for improved diagnosis so that the MRI operator can perform additional scans while the patient is still inside the scanner. This capability will also minimize the need to recall the patient for additional scans.

In the example of a patient specific double inversion recovery (DIR) pulse sequence, values for scan parameters may be optimized substantially in real time. In particular, in one embodiment the T1 and T2 relaxation times and the relative proton densities of the different tissues of a patient are mapped using attribute scout scan acquisitions, and the values of these parameters are used to optimize the tissue contrast on subsequent MR imaging or spectroscopy acquisitions. Scout scans for diffusion, perfusion, or geometry may be used in a similar fashion as the relaxation times to customize the scan for an individual patient.

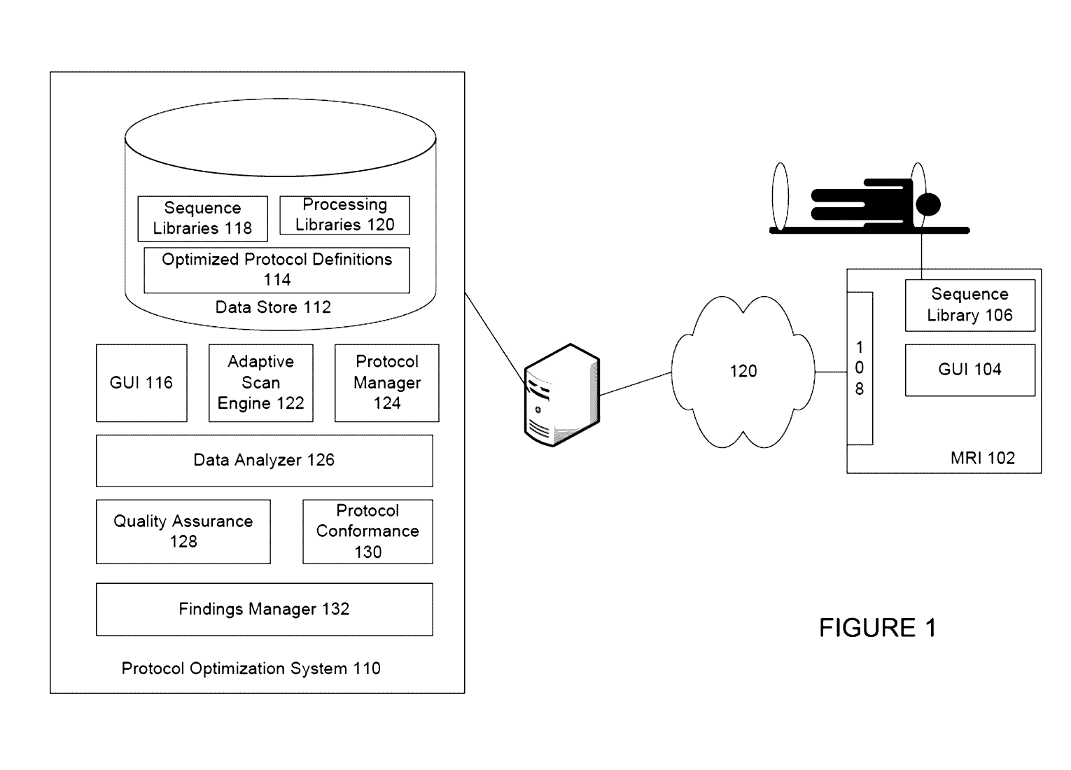

Turning now to FIG. 1, in one embodiment of a protocol optimization system 110 for real-time optimization, an MRI scanner 102 for scanning patients may implement a graphical user interface (GUI) 104 with which the MRI scanner operator may interact and a sequence library 106. The sequence library 106 may identify one or more sequences. Each scan within the sequence library 106 may include one or more pulse sequences for operating the MRI scanner 102 to produce a particular set of image or other data. Examples of such sequences or scans (which will be used interchangeably herein) include Dual-Echo Fast Spin Echo (FSE), Fluid Attenuated Inversion Recovery (FLAIR), Dual Inversion Recovery (DIR), etc. Each of the scans in the sequence library 106 may operate based on one or more scan parameters where the scan parameters may be chosen to optimize a particular objective. In a manual mode of operation an operator may select a scan from the sequence library 106 (e.g., from a drop-down menu of scan choices). Each selected scan may be associated with a set of scan parameters. Additionally, the operator may manually enter the scan parameter values of that scan through the GUI 104.

MRI scanner 102 may have an interface 108 which permits the scanner to be coupled over a network 120 to a protocol optimization system 110 such that data may be communicated between the MRI scanner 102 and the protocol optimization system 110. Such a network 120 may include a physical connector such as a serial or parallel connection, may be a wired or wireless interface and may, in one embodiment, communicate over a network 120 which may be a packetized communication network such as a local area network (LAN), a wide area network (WAN), the Internet, combinations of such networks, or other types of networks.

Additionally, it will be noted that while this embodiment has depicted the MRI scanner 102 and protocol optimization system 110 separately, in some embodiments the protocol optimization system 110 may be incorporated with MRI scanner 102. That is, functionality described herein for the protocol optimization system 110 may be implemented as part of the MRI scanner 102. In some embodiments, some of the functionality described herein may be implemented as machine instructions (e.g., software) executing on a computer resource such as one or more central processing units (CPUs), one or more computers, etc. Such a computer resource may be included with, or connected to the MRI scanner 102.

Protocol optimization system 110 includes a central processing unit (not shown) and a computer readable medium including instructions for use in implementing the protocol optimization system 110. In certain embodiments, the protocol optimization system 110 may include one or more dedicated graphical process units (GPU) which may process in parallel and which may process in parallel to the central processing unit (CPU). The use of such GPUs and parallel processing techniques in the protocol optimization system 110 may speed the processing of data received from the MRI scanner 102.

Protocol optimization system 110 may include a data store 112 (e.g., non-volatile storage such as a hard or solid state storage drive) that includes a set of optimized protocol definitions 114. Each optimized protocol definition 114 may define a set of MRI sequences and in a particular order. Each optimized protocol definition also may include or otherwise identify processing that should be performed on one or more of the scans. Such processing may include the analysis of the scan's data or determination of a parameter value using the scan's data. In one embodiment, a GUI 116 may be implemented within the protocol optimization system 110 to allow an operator to define an optimized protocol definition. For example, an operator may be presented with a representation of a set of scans from a sequence library 118 (e.g., sequences for imaging including T1-weighted, T2-weighted, FLAIR, double inversion recovery, magnetization transfer contrast, etc.) and a representation of processing blocks from processing libraries 120 (e.g., software executable modules for segmentation of the brain tissues, spatially registering different image sets, curve fitting to calculate tissue attributes such as relaxation times, relative proton density, etc.). A processing block may include machine instructions that are executed by a processor, computer, etc. The processing blocks may be executable by the protocol optimization system 110 to perform data analysis or parameter definitions. Thus, by selecting the appropriate representations of scans and processing blocks and linking together the selected scans and processing blocks, an operator may construct an optimized protocol definition that may be saved as an optimal protocol definition 114 for use by the protocol optimization system 110.

In the example of FIG. 1, the protocol optimization system 110 also may include an adaptive scan engine 122, a protocol manager 124, a data analyzer 126, quality assurance module 128, protocol conformance module 130 and findings manager 132. Each of these components may be implemented as machine instructions executing on a computer resource. In some embodiments, the components are implemented as separate components as shown in FIG. 1. In other embodiments, the functionality of two or more (or all) of the components may be implemented as a single set of machine instructions (component).

Data analyzer 126 may receive data from MRI scanner 102 through interface 108. Such data may include images acquired during a scan of the patient and/or other types of data (e.g., configuration or operational data) communicated from the MRI scanner 102. The data analyzer 126 may perform (substantially in real time) analysis of the data to determine quantitative and/or qualitative metrics associated with that data. Such metrics may include physical attributes of the tissue such as tissue T1 and T2 relaxation time values, and optimal values for scan parameters such as repetition time (TR), echo time (TE), flip angle (FA), etc. The data analyzer 126 may implement or otherwise have access to, for example, numerical solvers for performing curve fitting and function optimization tasks.

The protocol manager 124 may include instructions configured for using the data (e.g., as determined by data analyzer 126) to calculate some or all of the optimized scan parameter values noted above and/or additional optimized scan parameter values for use in a particular scan. For example, the protocol manager may calculate the flip angle that produces the maximum signal-to-noise ratio (SNR) for a sequence with a specific TR of a tissue for a particular measured T1—known as the Ernst angle. The protocol manager 124 may also include instructions for processing data determined (e.g., calculated) by data analyzer 126 to determine whether additional scans should be performed, whether scans should be removed from a currently running protocol or whether special actions (e.g., contrast injection, change of the scan plane, etc.) should be performed. These decisions may be made using a database with predefined rules.

The adaptive scan engine 122 may adjust the MRI scanner 102 according to the determination of the protocol manager 124. For example, the adaptive scan engine 122 may communicate with MRI 102 through interface 108 to specify a scan to be performed and provide parameter values to use with the specified scan. Additionally or alternatively, the adaptive scan engine 122 may interact with an interface (e.g., GUI 116 or 104) to permit an operator to specify the scan to be performed, parameters to use, additional scan(s) that should be considered, special actions that should be performed, etc.

Quality assurance module 128 may analyze the quality of the data (e.g., calculate the SNR ratio or other measures of data quality) and may generate an alert (e.g., audible alert, visual alert, etc.) whenever the metric of quality falls below a preconfigured threshold value for that metric or scan. The protocol conformance module 130 may analyze the data from the MRI scanner 102 (or results from data analyzer 126) and compare the data or scans to the protocol definition currently running to detect substantial deviations from the desired protocol. By inspecting the scan parameters in the DICOM header from the MRI scanner 102 and detecting a parameter (e.g., repetition time, TR, etc.) outside a predefined range, the module may detect departure from the prescribed protocol. Similarly, improper geometrical coverage for an image or a lack of the desired tissue contrast pattern for the image also may be designated as violations of the protocol. These violations can be detected by comparing values of the scan parameters to preconfigured thresholds, or by comparing the images to a template image with the desired features.

Findings manager 132 may analyze the data from MRI scanner 102 or data analyzer 126. The findings manager 132 may perform certain analyses of the images according to a specified protocol objective. An example of such an analysis is the identification of focal hyperintensity on T2-weighted images, which may indicate the presence of lesion activity. According to the rule recorded in a database included in or otherwise accessible to the findings manager 132, the detection of lesions may cause the findings manager 132 to promptly suggest to the operator of the MRI scanner 102 that one or more additional localized high resolution scans or one or more magnetic resonance spectroscopy acquisitions may be helpful, and automatically prescribe the geometrical coverage of the scan to match the detected abnormalities. Such suggestions may be displayed to the operator via a GUI such as GUI 104 or GUI 116.

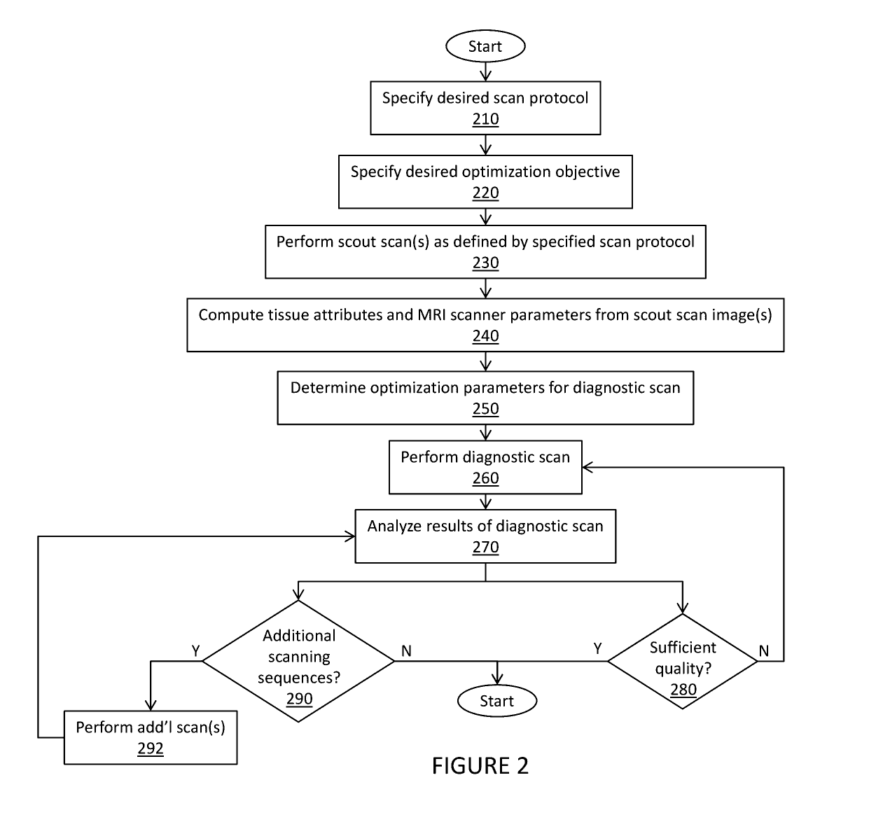

FIG. 2 shows an embodiment of a method for implementing a patient-specific MRI protocol for use with embodiments of the protocol optimization system. The operations may be performed in the order shown, or in a different order. Further, the operations may be performed sequentially, or two or more of the operations may be performed concurrently.

Initially, at 210 a desired scan protocol may be specified. Such a protocol may be specified by, for example, selection by an operator interacting with a GUI at the MRI scanner 102 or at the protocol optimization system 110. In one embodiment, an operator interacting with the MRI scanner may be presented the option to select an optimized protocol for a particular scan when an optimized protocol definition for such a scan has been defined in the protocol optimization system and stored in the optimized protocol definitions 114. For example, a diffusion-weighted scan may include the option to optimize the diffusion-weighting parameters (referred to as a b-value) that maximizes the accuracy and precision in the measured diffusion coefficient. The specified scan protocol may be received by the adaptive scan engine 122 of the protocol optimization system 110.

At 220, a desired optimization objective for the scan also may be specified and received by the adaptive scan engine 122. The specified optimization objective, for example, may be to maximize the contrast between two tissue types, minimize an image artifact (e.g., motion artifact), reduce the total scan duration, etc. The desired objective may be provided by, for example, a health care professional such as a radiologist to the MRI scanner operator who then may input the objective into the protocol optimization system 110 (e.g., through the interface of the MRI scanner, directly into an interface implemented by the protocol optimization system or into a single interface in cases where the protocol optimization system is integrated with the MRI scanner). The optimization objective may be selected from, for example, a drop down menu, a set of radio buttons, by typing in the objective into the user interface, etc. and input into the adaptive scan engine 122.

As explained above, an MRI protocol definition may be defined by a set of pulse sequences and the specified scan parameters of each sequence. The protocol definition for the desired scan may include one or more initial scans to be performed to aid in patient or machine specific data acquisition (i.e., the attribute scout scans), followed by a diagnostic scan to be performed with optimized parameter value(s) computed based on the results of one or more of the attribute scout scans. Each attribute scout scan may result in a lower resolution image than the diagnostic scan and thus be completed in less time than the subsequent diagnostic scan. Accordingly, at 230 the MRI scanner 102 performs the attribute scout scans defined by the scan protocol specified at 210. A single attribute scout scan may be performed or multiple scout scans may be performed in accordance with the specified protocol. In some embodiments, the adaptive scan engine 122 may send a signal or message to the MRI scanner 102 to initiate the desired attribute scout scans (in which case the MRI scanner automatically performs the attribute scout scans), or the MRI scanner operator may initiate the attribute scout scans that have been specified by the protocol optimization system 110 to the MRI scanner.

At 240, the resulting images from the attribute scout scans may be analyzed by, for example, the data analyzer 126 to measure patient tissue attributes (e.g., patient physiological parameters) or MRI scanner specific parameters. Examples of such computed tissue attributes may represent physical or MR tissue parameters such as proton density, diffusion coefficient, relaxation times (T1, T2, T2*, etc.), or any MR-measurable property of the tissues. The T1 attribute may be computed by performing a Look-Locker scan. A T2 attribute may be computed by performing a multi-echo spin echo scan. Proton density may be computed by performing a short-TE spin echo scan. A water diffusion coefficient may be computed by performing a diffusion tensor image. Magnetic susceptibility may be computed through performance of quantitative susceptibility mapping. In one embodiment, in addition to, or in lieu of, performing one or more initial attribute scout scans for the patient, patient specific data such as images from previously performed scans or other previously determined patient specific data may be provided to the protocol optimization system. For example, image files of previous patient scans may be uploaded or otherwise stored in the protocol optimization system in association with the patient. The attribute scout scans also may be used to compute scanner parameters. For example, the center frequency may be computed through performance of free induction decay scouting sequence. Static field homogeneity may be computed through performance of a dual-TE gradient echo sequence, and transmit field homogeneity may be computed through performance of an interleaved-TR fast gradient echo scan.

The patient attributes computed at step 240 may be processed at step 250 to determine values for the optimization parameter(s) for the subsequent diagnostic scan(s). In one embodiment, the parameters for the diagnostic scan that are to be optimized can be determined based on the specified diagnostic scan and optimization objective. The scan parameters to be optimized may be selected according to their influence on the objective function. For example, in inversion recovery sequences, the inversion time(s) and the echo time are the major control parameters that determine image contrast. In diffusion-weighted scans, the b-value parameter determines the degree of diffusion weighting in the image. Specifically, in one embodiment, data from an initial attribute scout scan, which may be a parameter scouting scan, may be processed according to a set of processing instructions defined by the optimized protocol definition to yield a metric or other value such as the average T1 for brain white matter (WM). In one particular embodiment, each of the attribute scout scans of the optimized protocol definition may have a set of associated processing blocks defined for it as part of the optimized protocol definitions 114, such that by processing the data acquired during the initial scan according to the processing blocks a metric associated with that initial scan may be determined. These processing blocks may be defined, for example, when the optimized protocol definition was defined with respect to the optimized protocol system. In certain embodiments, the processing of the data of the attribute scout scans may be processed in parallel using one or more graphics processing units (GPUs) or other processing cores or code. The parallel processing of such data may provide the speed of processing desired to limit the patient's time in the scanning device such that the desired optimized scan may be performed substantially in a single scanning session, reducing the patient's time in the scanner or to address other time related concerns.

The patient attributes determined through the processing of the data acquired in one or more of the initial attribute scout scans may be utilized to determine the optimal values of the scan parameters for the desired diagnostic scan based on the optimization objective for the scan. Once the values of the scan parameters for the diagnostic scan are determined, these scan parameters may be provided to the MRI scanner for use in conducting the diagnostic scan which is performed at step 260 using the optimized scan parameter values. The parameter may be transmitted to the scanner and then a signal asserted to the scanner to automatically initiate the diagnostic scan. Thus, the operator need not be involved in the control loop to input the parameters from the attribute scout scan(s) and trigger the scanner to perform the diagnostic scan(s). The adaptive scan engine 122 may transmit the computed optimized scan parameter values to the MRI scanner 102 for use in configuring the scanner during diagnostic scan. In this manner, the diagnostic scan can be performed on the patient using parameter values for that scan that have been substantially optimized for the actual patient undergoing that scan. As patient specific parameter values may be utilized, better results for the desired scan may be obtained.

Table I below provides examples of various diagnostic sequences, scan parameters that can be optimized, tissue attributes that can be measured and optimization functions that can be specified.

TABLE I
DX sequences, scan parameters, tissue
attributes, and optimization functions

| | | Tissue | |
|---|---|---|---|
| Example | Example Scan | Attributes | |
| Diagnostic | Parameters To | To Be | Example Optimization |
| Sequence | BE Optimized | Measured | Objective Function |
| FLAIR for lesions | TI, TR, TE | PD, T1, | CSF Suppression Level: |
| in central nervous | | T2 | S_{CSF}/(S_{GM} + S_{WM}) |
| system | | | Lesion-WM Contrast: |
| | | | (S_{Lesion} − S_{WM})/S_{WM} |
| | | | Lesion-CSF Contrast |
| | | | GM-WM Contrast |
| Fast Gradient | FA, TR | T1 | GM-WM Contrast |
| Echo for brain | | | |
| volumetry | | | |
| Double Inversion | TI1, TI2 | PD, T1, | CSF suppression |
| Recovery for GM | | T2 | WM suppression |
| volumetry/lesions | | | SNR of GM |
| Diffusion | b-value | ADC | Accuracy and precision |
| Weighted/Tensor | | | in measured ADC and |
| Imaging | | | derived diffusion metrics |
| for assessing | | | (mean diffusivity |
| WM integrity | | | fractional anisotropy) |
| | | | Accuracy of WM fiber |
| | | | tracks |
| MR Spectroscopy | TR, FA, TE, | T1, T2 | Metabolite SNR |
| for tumors | NEX, Matrix | Organ | Lipid contamination |
| | FOV, | geometry | |
| | Location of | Lipid | |
| | suppression | frequency | |
| | bands | | |

As an example the step-by-step procedure for optimizing Fluid-Attenuated Inversion Recovery (FLAIR) brain scans is described below. The FLAIR sequence suppresses the signal from fluids (e.g., cerebrospinal fluid (CSF) in the brain) and is typically used to visualize lesions in the brain and spinal cord. To optimize the FLAIR sequence for a particular patient, the scan protocol may run a fast T1-mapping sequence such as the Look-Locker or a variable-flip-angle sequence. Mapping of T2 and proton density (PD) can be similarly performed using a multi-echo sequence. The data from these two sequences are then analyzed to calculate values of PD, T1 and T2 at each voxel in the imaged volume. Calculation of these tissue parameters can be done analytically or numerically by the data analyzer 126 by fitting the collected data to the signal model. For example, PD and T2 are determined by fitting the data at various echo times (TE₁, TE₂, etc.) as Signal(TEᵢ)=PD×exp(−TEᵢ/T2), i=1,2, . . . . An appropriate fitting algorithm such as the Levenberg-Marquardt may be used. The computed (PD, T1, T2) values of all tissues in the brain (white matter, gray matter (GM) lesions, etc.) are then used by, for example, the protocol manager 124, to select the scan parameters of the FLAIR sequence. The parameters that may influence the FLAIR sequence the most are the inversion time (TI), echo time (TE) and repetition time (TR). These parameters can be analytically or numerically optimized by the data analyzer 126 or protocol manager 124 to maximize an objective function. For imaging brain lesions, the objective function may be defined, for example, as the contrast between the lesions and the brain parenchyma, expressed as Contrast={Sₗₑₛᵢₒₙ−S_{brain}}/S_{brain}, where S denotes the MR signal at the voxel, and S typically depends on both the properties of the tissues (PD, T1, T2), and on the scan parameters. Optimizing the scan parameters may be based on representative values of the tissue attributes (e.g., average values), or may vary on a slice-by-slice basis.

In one example, the scan parameters to be optimized for DIR may comprise TI1 (first inversion time) and TI2 (second inversion time). One or more attribute scout scans may be performed for the system to measure T1 (longitudinal relaxation time of WM, GM and CSF, T2 (transverse relaxation time of WM, GM and CSF), and PD (proton density of WM, GM and CSF). The objective function to be optimized may be to minimize Ψ={S_{WM}+S_{CSF}}/S_{GM}. Sₜᵢₛₛᵤₑ denotes the MR signal generated by tissue at the current values of the scan parameters. The signal S depends on both the tissue attributes and the scan parameters: Sₜᵢₛₛᵤₑ=f(TI1, TI2; T1ₜᵢₛₛᵤₑ, T2ₜᵢₛₛᵤₑ, PDₜᵢₛₛᵤₑ). The function f depends on the pulse sequence, which for conventional DIR is:

f(TI1,TI2;T1,T2,PD)=PD×exp(−TE/T2)·{1−2·exp(−TI2/T1)+2·exp(−[TI1+TI2]/T1)−exp (−TR/T1)}

Modifications in the pulse sequences may require corresponding modification to the function f to account for differences in the inversion flip angles or use of fast spin echo readout modules. Finally, in this example, a solver (processing block) is invoked within the system to compute the scan parameters for:

Minimize Ψ={f(TI1,TI2;T1_{WM},T2_{WM},PD_{WM})+f(TI1,TI2;T1_{CSF},T2_{CSF},PD_{CSF})}/f(TI1,TI2;T1_{GM},T2_{GM},PD_{GM})

Once the diagnostic scan with the optimized parameter values has been performed (260), the results (e.g., image data) from the diagnostic scan may be analyzed at 270 by, for example, the protocol manager 124, data analyzer 126, quality assurance module 128 and/or protocol conformance module 130. In one embodiment, the analysis may determine at 280 whether the resulting image is of sufficient diagnostic accuracy or quality to be useful to a clinician. Such an analysis may be performed by the quality assurance module 128 and may be based on a computed signal to noise ratio (SNR) or detected artifacts within the image data, or other image quality criteria. This step is task-specific, and may require the determination of suitable image quality measures and tolerance limits. In addition, access to proper data in the reconstruction pipeline may be useful.

For example, with the widespread use of phased array coils and parallel imaging strategies, the statistical properties of the noise are changed and spatially varying. Estimation of the noise level requires accounting for these effects by selecting a suitable region of interest and including a suitable statistical noise model. Moreover, the background in these images may be masked out during reconstruction, complicating the estimation of noise and artifact levels. Access to intermediate images in the reconstruction pipeline allows the measurement of noise and image artifacts. If the data is not of sufficient quality the quality assurance module 128 may request or suggest that the diagnostic scan may be performed again, at which point control loops back to 260 in FIG. 2.

Criteria for the image quality include the image SNR, which can be measured by the quality assurance module 128 from an estimate of the signal in a homogenous part of the image divided by the standard deviation of the noise measured in the background, or estimated from the object itself in case the background is masked. Another image quality criterion is the level of artifacts in the image which may result from system imperfection or from patient motion. Signal ghosting may be detected in the background where signal is not expected. The intensity of the background signal is then used as an indicator of the level of the artifacts. The artifact voxels can be obtained by histogram thresholding to separate out noise from ghosting (see e.g., Mortamet et al. Magnetic Resonance in Medicine 62.2 (2009): 365-372). The artifacts often appear as a ghosting signal that propagates into the image background. Detecting background signal above the noise level may be used as one criterion for identifying corrupted scans. The background of an image can be identified using a registration technique to a suitable template. Other criterion for image quality includes a measure such as low contrast-to-noise ratio (CNR) between two tissues, or weak suppression of interfering signal (e.g., insufficient fat saturation).

If automated analysis indicates insufficient image quality, the operator may be alerted to the problematic images and given the option to repeat the scan. Information on the possible cause of the reduced image quality and recommended actions to improve the image quality by adjusting the scan parameters may be suggested through the appropriate GUI. The algorithms for detecting these artifacts exploit basic MRI physics. For example, the presence of ghosting is often the result of patient motion, incorrectly prescribed field-of-view, or a very high acceleration factor when using parallel imaging. A bright spot in the image space may indicate radio-frequency leak in the MR room shielding, and so on.

The data resulting from the initial attribute scout scans or the diagnostic scan also may be analyzed at 290 to determine whether additional diagnostic scanning sequences should be performed. If it is determined that additional scans should be performed, these additional scans can be performed at step 292 by initiating the scan through a communication with the MRI scanner or by notifying an operator. This process may be repeated until all diagnostics scans are performed. In this manner, not only can individual desired scans be optimized for a particular patient, but in addition, the entire scanning process can be optimized by determining, in substantially real time, additional scans to be performed on a patient based on results obtained in previous scans from that patient. One example is using automated image segmentation to detect signal abnormalities that could represent a lesion or tumor. In this case, to augment conventional MRI sequences, a contrast-enhanced MRI acquisition may be suggested to provide further information about the suspected mass. Another situation is when suspected abnormalities are detected on axially-acquired images of the brain near the corpus callosum of close to the air-tissue interface at the base of the brain. Such situation suggests the addition of a sagittal acquisition for a better view of the suspected abnormalities. The rules and recommended actions are saved as a list, and are accessed during execution using logic operations.

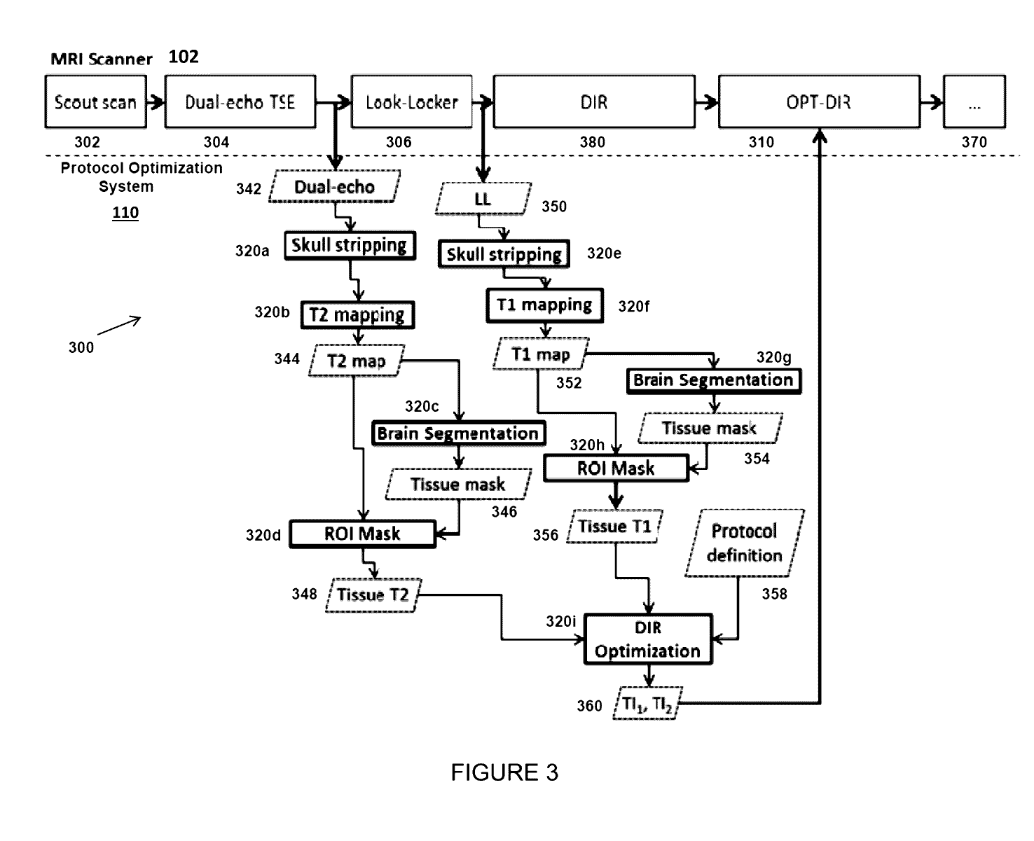

FIG. 3 depicts an example of a process flow of an embodiment of an optimized protocol for the adaptive and patient-specific optimization of the double inversion recover (DIR) scan. Optimized DIR protocol 300 may include an optimized DIR scan 310 and a set of initial attribute scout scans which include geometry scout scan 302, dual-echo TSE scan 304 and look-locker scan 306. For reference, a DIR scan 380 is included in this example, but is included only as a reference and may not be a part of the optimized DIR protocol. The optimized DIR protocol 300 may also include a set of processing blocks 320a-320i which specify how data from the initial scans is to be processed, such as in what order is the data is to be processed, what data is computed, etc. Processing blocks 320a-320i may be included as part of, or referenced in, the optimized protocol definitions 114 as associated with the scan to be performed as noted above. Some or all of these processing blocks may be implemented, for example, by the data analyzer 126.

Initially the operator may specify that an optimized DIR scan is desired for a patient and also specify as an optimization objective that the contrast between white matter and grey matter is to be optimized as well as optimize the separation between white matter and cerebrospinal fluid (CSF). The protocol optimization system 110 may access the optimized protocol definition for an optimized DIR scan, and determine that the first scan in the optimized DIR protocol definition is a attribute scout scan and communicate with the MRI scanner 340 to initiate the attribute scout scan. When the attribute scout scan is complete and MRI scanner 102 is ready to perform another scan, the protocol optimization system may initiate the dual-echo TSE scan 304. The dual-echo turbo-spin echo (TSE) scan 304 may be performed by the MRI scanner 340 according to its standard definition of a dual-echo TSE scan (e.g., according to standard parameters for the dual-echo TSE scan).

When the dual-echo TSE scan 304 is complete, the resulting dual-echo image data 342 may be provided to, or otherwise obtained by, the protocol optimization system 110. This image data 342 may be processed using skull stripping block 320a to extract the brain tissue from the image data 342. The resulting data from the two images after being skull stripped by skull stripping block 320a may be processed by T2 mapping block 320b to acquire the T2 map 344 for the patient (e.g., using fitting to a monoexpoenential curve). The T2 map 344 may be processed by brain segmentation processing block 320c to separate the grey matter from the white matter to obtain tissue mask 346. This tissue mask 346 along with the T2 map 344 may be processed by Region of Interest (ROI) mask processing block 320d to obtain representative T2 values 348 for the individual tissues of the patient.

When the dual-echo TSE scan 304 is complete and MRI scanner 340 is ready to perform another scan, the protocol optimization system 330 may initiate the Look-Locker scan 306. The Look-Locker scan 306 may be performed by the MRI scanner 340 according to its standard definition of a look-locker scan (e.g., according to standard parameters for the look-locker scan 306). It will be noted that the look-locker scan 306 may be initiated by protocol optimization system substantially immediately after the conclusion of the dual-echo TSE scan 304 such that data from the dual-echo TSE scan 342 may be processed, before, while, or after Look-Locker scan 306 is being conducted.

When the Look-Locker scan 306 is complete, the resulting Look-Locker scan 306 image data 350 may be provided to, or otherwise obtained by, the protocol optimization system 330. This image data 350 may be processed using skull stripping block 320e to extract tissue data from the image data 350. The resulting data from the image after being skull stripped by skull stripping block 320e may be processed by T1 mapping block 320f to acquire the T1 map 352 for the patient. The T1 map 352 may be processed by brain segmentation processing block 320g to separate the grey matter from the white matter to obtain tissue mask 354. This tissue mask 354 along with the T1 map 352 may be processed by Region of Interest (ROI) mask processing block 320h to obtain representative T1 values 356 for the patient.

The determined T2 values 348, T1 values 356 and optimization objective 358 for the desired protocol may be processed by the DIR optimization processing block 320i. DIR optimization processing block 320i may use the determined T2 values 348 and T1 values 356 for the patient and the optimization objective 358 to determine optimized inversion parameters values TI₁ and TI₂ 360 for the patient to maximize the gray matter-white matter contrast, using a suitable optimization procedure. The determined TI₁ and TI₂ values 360 for patient may be provided to the MRI scanner 330 to use for performing the desired DIR scan 310. In this manner, the DIR scan of the MRI scanner 330 may be converted to an optimized DIR scan 310 and substantially optimized results obtained for the patient. Following this optimized DIR scan 310, the scan may be repeated if the quality of data is insufficient for diagnostic analysis or additional scans 370 may be performed if an analysis of data resulting from the DIR scan determines that additional scans would be diagnostically useful.

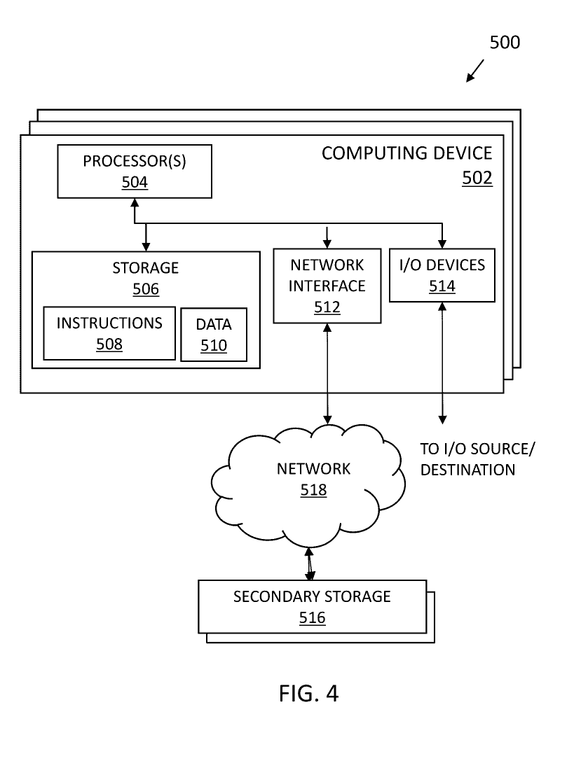

FIG. 4 shows a schematic diagram for a computing system 500 suitable for implementation of the protocol optimization system 110 including, for example, the GUI 116, adaptive scan engine 122, a protocol manager 124, a data analyzer 126, quality assurance module 128, protocol conformance module 130 findings manager module 132 as well as the data store 112 in accordance with various embodiments. The system includes one or more computing devices 502. The computing system 500 includes the computing devices 502 and secondary storage 516 communicatively coupled together via a network 518. One or more of the computing devices 502 and associated secondary storage 516 may be used to provide the functionality of the various components, processing blocks, devices, and databases described herein.

Each computing device 502 includes one or more processors 504 coupled to a storage device 506, network interface 512, and I/O devices 514. In some embodiments, a computing device 502 may implement the functionality of more than one component of the system 100. In various embodiments, a computing device 502 may be a uniprocessor system including one processor 504, or a multiprocessor system including several processors 504 (e.g., two, four, eight, or another suitable number). Processors 504 may be any suitable processor capable of executing instructions. For example, in various embodiments, processors 504 may be general-purpose or embedded microprocessors implementing any of a variety of instruction set architectures (“ISAs”), such as the x86, PowerPC, SPARC, or MIPS ISAs, or any other suitable ISA. In multiprocessor systems, each of processors 504 may, but not necessarily, commonly implement the same ISA. Similarly, each of the computing devices 502 may implement the same ISA, or individual computing nodes and/or replica groups of nodes may implement different ISAs.

The storage device 506 may include a non-transitory, computer-readable storage device configured to store program instructions 508 and/or data 510 accessible by processor(s) 504. The storage device 506 also may be used to store the machine images as explained above. The storage device 506 may be implemented using any suitable volatile memory (e.g., random access memory), non-volatile storage (magnetic storage such as a hard disk drive, optical storage, solid storage, etc.). Program instructions 508 and data 510 implementing the functionality disclosed herein are stored within storage device 506. For example, instructions 508 may include instructions that when executed by processor(s) 504 implement the various modules and/or other components of the protocol optimization system 110 disclosed herein.

Secondary storage 516 may include additional volatile or non-volatile storage and storage devices for storing information such as program instructions and/or data as described herein for implementing the various aspects of the service provider's network described herein. The secondary storage 516 may include various types of computer-readable media accessible by the computing devices 502 via the network 518. A computer-readable medium may include storage media or memory media such as semiconductor storage, magnetic or optical media, e.g., disk or CD/DVD-ROM, or other storage technologies. Program instructions and data stored on the secondary storage 516 may be transmitted to a computing device 502 for execution by a processor 504 by transmission media or signals via the network 518, which may be a wired or wireless network or a combination thereof. Each of the interactive content generator 100, the script generator 110, and the digital content generator 120 as well as the various databases and other components described herein may be implemented as a separate computing device 502 executing software to provide the computing node with the functionality described herein. In some embodiments, some or all of the various functional components may be implemented by the same computing device.

The network interface 512 may be configured to allow data to be exchanged between computing devices 502 and/or other devices coupled to the network 518 (such as other computer systems, communication devices, input/output devices, or external storage devices). The network interface 512 may support communication via wired or wireless data networks, such as any suitable type of Ethernet network, for example; via telecommunications/telephony networks such as analog voice networks or digital fiber communications networks; via storage area networks such as Fibre Channel SANs, or via any other suitable type of network and/or protocol.

Input/output devices 514 may include one or more display terminals, keyboards, keypads, touchpads, mice, scanning devices, voice or optical recognition devices, or any other devices suitable for entering or retrieving data by one or more computing devices 502. Multiple input/output devices 514 may be present in a computing device 502 or may be distributed on various computing devices 502 of the system 500. In some embodiments, similar input/output devices may be separate from computing device 502 and may interact with one or more computing devices 502 of the system 500 through a wired or wireless connection, such as over network interface 512.

Any suitable programming language can be used to implement the routines, methods or programs of embodiments of the invention described herein, including C, C++, Java, assembly language, etc. Different programming techniques can be employed such as procedural or object oriented. Any particular routine can execute on a single computer processing device or multiple computer processing devices, a single computer processor or multiple computer processors. Such a computer may include a display and input device such as a keyboard and mouse or track pad. In some embodiments, the computing resource is integrated into the MRI scanner 102. Data may be stored in a single storage medium or distributed through multiple storage mediums, and may reside in a single database or multiple databases (or other data storage techniques). The sequence of operations described herein can be interrupted, suspended, or otherwise controlled by another process, such as an operating system, kernel, etc. The routines can operate in an operating system environment or as stand-alone routines. Functions, routines, methods, steps and operations described herein can be performed in hardware, software, firmware or any combination thereof.

The embodiments described herein can be implemented as machine instructions stored on a computer-readable storage medium and executable by a computing resource such as one or more CPUs, one or more computers, etc. When executed, the machine instructions cause the computing resource to perform some or all of the operations described herein. A computer-readable medium may be any medium that can contain or store the machine instructions for execution by the computing resource. The computer readable medium can be, by way of example, an electronic, magnetic, optical, electromagnetic, infrared, or semiconductor system, apparatus, system, device, or computer memory.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited to only those elements but may include other elements not expressly listed or inherent to such process, process, article, or apparatus. Further, unless expressly stated to the contrary, “or” is inclusive not exclusive. For example, a condition A or B is satisfied by any one of the following: A is true (or present) and B is false (or not present), A is false (or not present) and B is true (or present), and both A and B are true (or present).

Although embodiments of the invention have been described with respect to specific embodiments thereof, these embodiments are merely illustrative, and not restrictive of the invention. The description herein of illustrated embodiments of the invention is not intended to be exhaustive or to limit the invention to the precise forms disclosed herein (and in particular, the inclusion of any particular embodiment, feature or function is not intended to limit the scope of the invention to such embodiment, feature or function). Rather, the description is intended to describe illustrative embodiments, features and functions in order to provide a person of ordinary skill in the art context to understand the subject matter without limiting the disclosure to any particularly described embodiment, feature or function. While specific embodiments of, and examples for, the invention are described herein for illustrative purposes only, various equivalent modifications are possible within the spirit and scope of the invention, as those skilled in the relevant art will recognize and appreciate. For example, while the illustrative embodiments depict MRI imaging, this system could also be implemented with other computer aided imaging methodologies. As indicated, these modifications may be made in light of the foregoing description of illustrated embodiments of the invention and are to be included within the spirit and scope of the disclosure and claims.

What is claimed is:

1. A system, comprising:
a magnetic resonance imaging (MRI) scanner; and
a protocol optimization system including a data store containing a plurality of optimized protocol definitions, a storage device containing executable machine instructions, and a computing resource configured to execute the machine instructions, wherein the protocol optimization system is configured to:
based on an input from a user interface of an optimized protocol definition, access the data store to identify a attribute scout MRI scan, a processing block mapped to the identified attribute scout MRI scan, and a diagnostic scan;
provide an identity of the identified attribute scout MRI scan to the MRI scanner;
using the processing block, analyze a resulting image from the MRI scanner upon performance of the attribute scout MRI scan to compute a patient tissue attribute and, using the computed patient tissue attribute, an optimized parameter value for the identified diagnostic scan;
automatically provide the optimized parameter value to the MRI scanner and assert a signal to the MRI scanner to perform a diagnostic scan using the optimized parameter value.

2. The system of claim 1, wherein the patient tissue attribute includes at least one of a T1 relaxation time, a T2 relaxation time, and a relative proton density, and the protocol optimization system is configured to use the patient tissue attribute and an objective function to compute at least one of an inversion time value, an echo time value, a repetition time value, and a flip angle.

3. The system of claim 2, wherein the protocol optimization system implements a user interface through which the objective function is received.

4. The system of claim 1, wherein the protocol optimization system is configured to compute a quality metric of an image resulting from the diagnostic scan.

5. The system of claim 4, wherein the quality metric includes at least one of a signal-to-noise ratio, an image-to-ghost ratio, and a geometric distortion value.

6. The system of claim 4, wherein the protocol optimization system is configured to determine that the quality metric is outside of an acceptable range and to generate a recommendation via a user interface for an additional diagnostic scan to be performed.

7. The system of claim 1, wherein the protocol optimization system is configured to perform an automated image process on an image resulting from the diagnostic scan and to generate a recommendation via a user interface as to whether an additional diagnostic scan is to be performed.

8. A system, comprising:
a protocol optimization system including a data store containing a plurality of optimized protocol definitions, a storage device containing executable machine instructions, and a computing resource configured to execute the machine instructions, wherein the protocol optimization system is configured to:
based on an input from a user interface of an optimized protocol definition, access the data store to identify a attribute scout magnetic resonance image (MRI) scan for an MRI scanner, a processing block mapped to the identified attribute scout MRI scan, and a diagnostic scan;
provide an identity of the identified attribute scout MRI scan to the MRI scanner;
using the processing block, analyze a resulting image from the MRI scanner upon performance of the attribute scout MRI scan to compute a patient tissue attribute and, using the computed patient tissue attribute, an optimized parameter value for the identified diagnostic scan;
automatically provide the optimized parameter value to the MRI scanner and assert a signal to the MRI scanner to perform a diagnostic scan using the optimized parameter value.

9. The system of claim 8, wherein the patient tissue attribute includes at least one of a T1 relaxation time, a T2 relaxation time, and a relative proton density, and the protocol optimization system is configured to use the patient tissue attribute and an objective function to compute at least one of an inversion time value, an echo time value, a repetition time value, and a flip angle.

10. The system of claim 9, wherein the protocol optimization system implements a user interface through which the objective function is received.

11. The system of claim 8, wherein the protocol optimization system is configured to compute a quality metric of an image resulting from the diagnostic scan.

12. The system of claim 11 wherein the quality metric includes at least one of a signal-to-noise ratio, an image-to-ghost ratio, and a geometric distortion value.

13. The system of claim 11, wherein the protocol optimization system is configured to determine that the quality metric is outside of an acceptable range and to generate a recommendation via a user interface for an additional diagnostic scan to be performed.

14. The system of claim 8, wherein the protocol optimization system is configured to perform an automated image process on an image resulting from the diagnostic scan and to generate a recommendation via a user interface as to whether an additional diagnostic scan is to be performed.
